(19) United States
(12) Patent Application Publication
(10) Pub. No.: US 2024/0375093 A1
(43) Pub. Date: Nov. 14, 2024

(54) PHOTOCATALYST SHEET

(71) Applicant: NIPPON TELEGRAPH AND TELEPHONE CORPORATION, Tokyo (JP)

(72) Inventors: Asuka MIYAKE, Musashino-shi, Tokyo (JP); Hitoshi SESHIMO, Musashino-shi, Tokyo (JP); Ryuji YAMAMOTO, Musashino-shi, Tokyo (JP); Tomohiro TANIGUCHI, Musashino-shi, Tokyo (JP); Ayako IWAKI, Musashino-shi, Tokyo (JP); Satoshi NARIKAWA, Musashino-shi, Tokyo (JP); Takahito KIRIHARA, Musashino-shi, Tokyo (JP); Kazuhide NAKAJIMA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP); Nobutomo HANZAWA, Musashino-shi, Tokyo (JP)

(73) Assignee: NIPPON TELEGRAPH AND TELEPHONE CORPORATION, Tokyo (JP)

(21) Appl. No.: 18/690,903

(22) PCT Filed: Sep. 24, 2021
(86) PCT No.: PCT/JP2021/035028

§ 371 Date: Mar. 11, 2024

(51) Int. Cl. B01J 35/39; A61L 2/10; B01J 35/54

(52) U.S. Cl. CPC B01J 35/39; A61L 2/10; B01J 35/54

(57) ABSTRACT

An object of the present invention is to provide a photocatalytic sheet configured to perform disinfection independent of the state of external light.

A photocatalytic sheet 50 according to the present invention includes a sheet 10 having a photocatalytic layer 17 on one surface, and an optical fiber 20 embedded in the sheet 10 and configured to supply ultraviolet light UV from inside of the sheet 10 to the photocatalytic layer 17. In the photocatalytic sheet, ultraviolet light is supplied from the optical fiber disposed in the sheet to the photocatalytic layer coated on the surface of the sheet (ultraviolet light is supplied from the back of the photocatalytic layer). This configuration allows for disinfection independent of the state of external light.

### TECHNICAL FIELD

The present disclosure relates to a photocatalytic sheet configured to perform sterilization and inactivate viruses by photocatalytic reactions with ultraviolet light.

### BACKGROUND ART

To prevent infectious diseases or the like, there has been a growing demand for a system that performs sterilization and inactivation of viruses using ultraviolet light. In the present embodiment, the word “disinfection” includes sterilization and inactivation of viruses.

When light (ultraviolet light) strikes the surface of titanium oxide, which is a photocatalyst, and a reaction occurs, two types of active oxygen O₂⁻ (superoxide ion) and —OH (hydroxyl radical) are generated by the reaction. The active oxygen decomposes organic substances in the air, leading to disinfection of the air, inactivation of viruses, and air deodorization (see, for example, Non Patent Literature 1). In order to cause this photocatalytic reaction, the surface of the substance needs to be coated with titanium oxide and irradiated with a light beam including ultraviolet light in the UV-A band (wavelength of 300 to 400 nm).

### CITATION LIST

### Non Patent Literature

Non Patent Literature 1: Photocatalysis Industry Association of Japan website (https://www.piaj.gr.jp/roller/contents/entry/200706118), searched on Apr. 19, 2021

### SUMMARY OF INVENTION

### Technical Problem

Most of the conventional photocatalytic products use sunlight or illumination light as a light source of UV-A band ultraviolet light necessary for reaction. Such conventional photocatalytic products sometimes fail to receive a sufficient amount of light (UV-A ultraviolet light dosage) required for photocatalytic reactions depending on the conditions such as the weather, sunlight, or shadow, resulting in insufficient disinfection performance.

The present invention has been made in view of the foregoing and it is an object of the present invention to provide a photocatalytic sheet configured to perform disinfection independent of the state of external light.

### Solution to Problem

To achieve the object above, a photocatalytic sheet according to the present invention includes an optical fiber to uniformly supply ultraviolet light to a coated photocatalytic layer. An ultraviolet lamp or an LED included in conventional ultraviolet light irradiation sources has a diameter of several millimeters to several centimeters, whereas an optical fiber has a much smaller diameter. By using the optical fiber as an ultraviolet light irradiation source, the ultraviolet light irradiation source can be installed in, for example, a sheet.

Specifically, the photocatalytic sheet according to the present invention includes a sheet having a photocatalytic layer on one surface, and an optical fiber embedded in the sheet and configured to supply ultraviolet light from inside of the sheet to the photocatalytic layer.

In the photocatalytic sheet, ultraviolet light is supplied from the optical fiber disposed in the sheet to the photocatalytic layer coated on the surface of the sheet (ultraviolet light is supplied from the back of the photocatalytic layer). This configuration allows for disinfection independent of the state of external light. The present invention, therefore, can provide a photocatalytic sheet configured to perform disinfection independent of the state of external light.

The optical fiber of the photocatalytic sheet according to the present invention may emit part of the propagating ultraviolet light from a side surface.

The optical fiber of the photocatalytic sheet according to the present invention may be configured to emit propagating ultraviolet light from an end, and may further include a turning function embedded in the sheet and configured to direct the ultraviolet light emitted from the end of the optical fiber toward the photocatalyst layer.

Note that each of the above inventions can be combined in any possible manner.

### Advantageous Effects of Invention

The present invention can provide a photocatalytic sheet configured to perform disinfection independent of the state of external light.

### BRIEF DESCRIPTION OF DRAWINGS

FIG. 1 is a diagram illustrating a photocatalytic sheet according to the present invention.

FIG. 2 is a diagram illustrating the photocatalytic sheet according to the present invention.

FIG. 3 is a diagram illustrating a light irradiation system including the photocatalytic sheet according to the present invention.

FIG. 4 is a diagram illustrating the light irradiation system including the photocatalytic sheet according to the present invention.

FIG. 5 is a diagram illustrating a light irradiation system including the photocatalytic sheet according to the present invention.

FIG. 6 is a diagram illustrating the light irradiation system including the photocatalytic sheet according to the present invention.

FIG. 7 is a diagram illustrating the photocatalytic sheet according to the present invention.

### DESCRIPTION OF EMBODIMENTS

The following describes embodiments of the present invention with reference to the accompanying drawings. The embodiments described below are examples of the present invention, and the present invention is not limited to the following embodiments. The same reference signs in the present description and the drawings denote the same components.

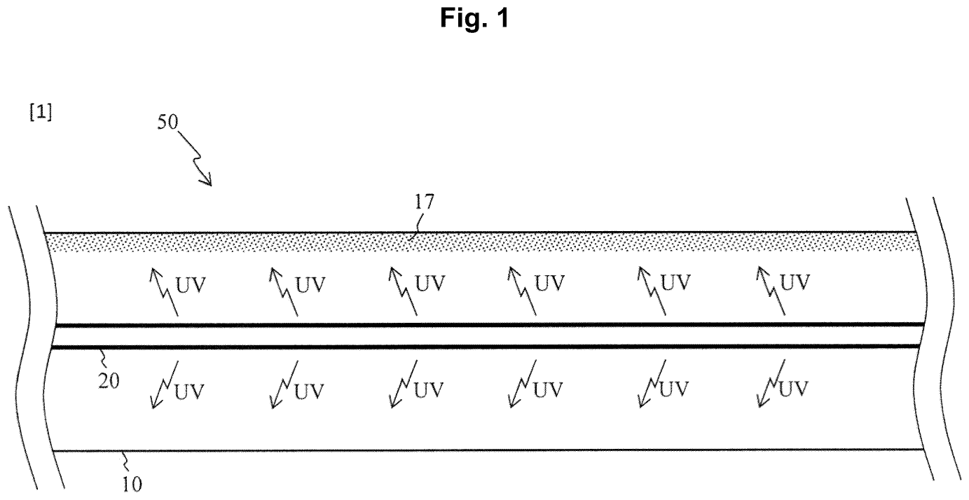

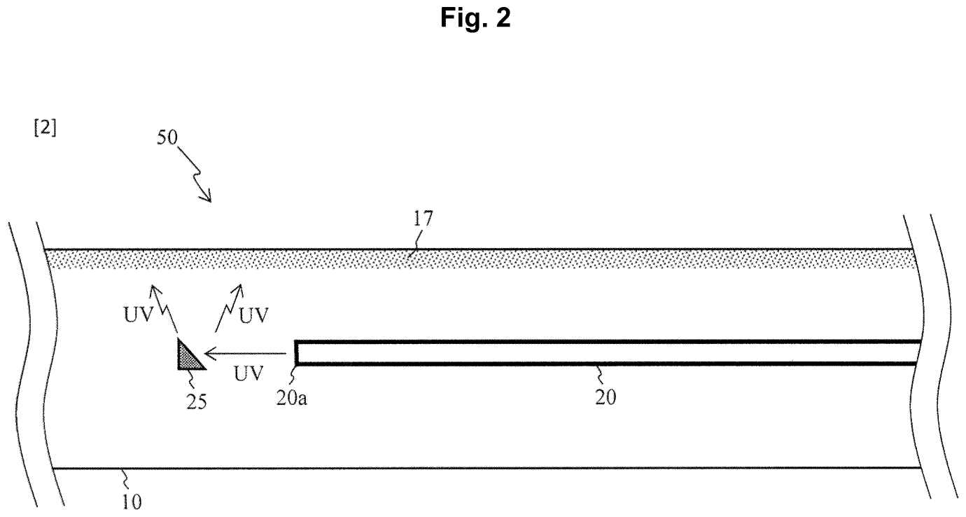

FIGS. 1 and 2 are diagrams illustrating a photocatalytic sheet 50 according to the present embodiment. The photocatalytic sheet 50 includes a sheet 10 having a photocatalytic layer 17 on one surface, and an optical fiber 20 embedded in the sheet 10 and configured to supply ultraviolet light from inside of the sheet 10 to the photocatalytic layer 17. The ultraviolet light is, for example, a light beam including ultraviolet light in the UV-A band (wavelength of 300 to 400 nm).

For example, the sheet 10 is made of resin, and has a structure in which the optical fiber 20 is embedded in the resin. The photocatalytic layer 17 is made of, for example, titanium oxide, and is coated on one surface of the sheet 10. In the present description, a surface of the photocatalytic layer 17 close to the optical fiber 20 is referred to as a “back surface of the photocatalytic layer 17”, and the opposite surface thereof is referred to as a “top surface of the photocatalytic layer 17”.

The optical fiber 20 of the photocatalytic sheet 50 in FIG. 1 emits part of the propagating ultraviolet light from a side surface. The optical fiber 20 is designed to promote leakage of the propagating light from the side surface. For example, the optical fiber 20 includes a scattering material in the core. The light propagating through the core is scattered by the scattering material, and the scattered light is emitted from the side surface through the cladding.

The ultraviolet light emitted from the side surface of the optical fiber 20 reaches the back surface of the photocatalytic layer 17 in the sheet 10. As described above, in order to cause a photocatalytic reaction, ultraviolet light needs to reach the top surface of photocatalytic layer 17. Thus, the photocatalytic layer 17 has a thickness that allows transmission of ultraviolet light. With the photocatalytic layer 17 having an appropriate thickness, two types of active oxygen, O₂⁻ (superoxide ion) and —OH (hydroxyl radical), are generated by the photocatalytic reaction, and the active oxygen decomposes organic substances in the air, thereby enabling disinfection.

It is preferred that the surface of the sheet 10 opposite to the photocatalytic layer 17 is shielded from light. If not, ultraviolet light is also emitted from the surface of the sheet 10 opposite to the photocatalytic layer 17. As described later, when the photocatalytic sheet 50 is disposed on a top surface of a table or the like, the top surface of the table is constantly irradiated with ultraviolet light, which may deteriorate the surface. Shielding the surface of the sheet 10 opposite to the photocatalytic layer 17 from light can prevent the ultraviolet light from causing degradation.

The surface of the sheet 10 opposite to the photocatalytic layer 17 may not necessarily be a light-shielding surface but may be a reflective surface that reflects ultraviolet light. The ultraviolet light emitted from the side surface of the optical fiber 20 to the surface opposite to the photocatalytic layer 17 of the sheet 10 is reflected by the reflective surface and can reach the photocatalytic layer 17. This configuration can save energy, or in other words, if the amount of ultraviolet light supplied to the optical fiber 20 is small, the effect of disinfection by photocatalytic reactions can be maintained.

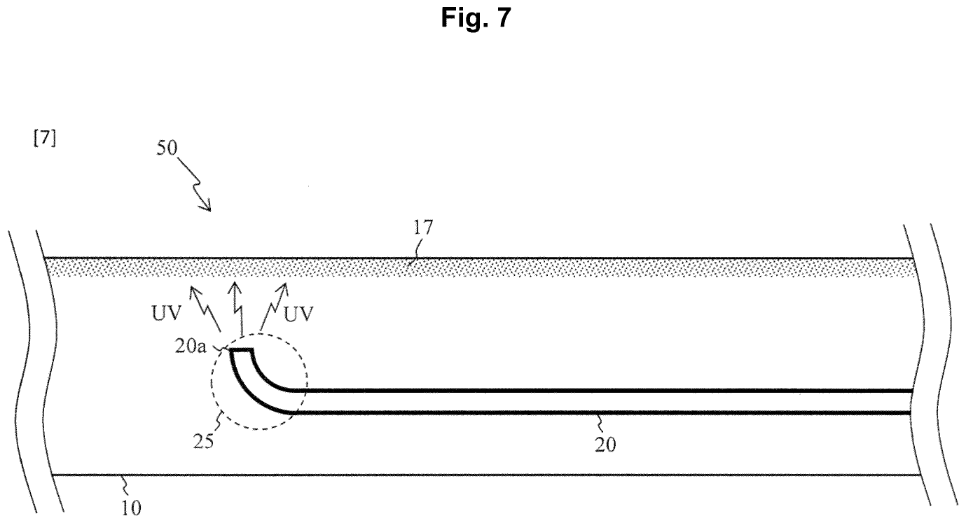

The photocatalytic sheet 50 may be configured as illustrated in FIG. 2 or 7. The optical fiber 20 of the photocatalytic sheet 50 in FIG. 2 or 7 emits propagating ultraviolet light from an end 20a, and further includes a turning function 25 embedded in the sheet 10 and configured to direct the ultraviolet light emitted from the end 20a of the optical fiber toward the photocatalyst layer 17.

The turning function 25 is, for example, a prism that scatters ultraviolet light in a plurality of directions as illustrated in FIG. 2. Alternatively, as illustrated in FIG. 7, the turning function 25 may be a formed portion at an end portion of the optical fiber 20 that is bent perpendicularly to the surface of the sheet 10 so that the ultraviolet light can be emitted to the photocatalytic layer 17 perpendicularly. In this case, it is preferred that the surface of the end 20a is formed in a scattering shape (for example, uneven surface) that scatters the ultraviolet light in a plurality of directions. With the turning function 25, the photocatalytic sheet 50 in FIGS. 2 and 7 need no light-shielding or reflective surface described above.

The photocatalytic sheet 50 may have a combined structure of the structure of FIG. 1 and the structure of FIG. 2.

### EXAMPLE 1

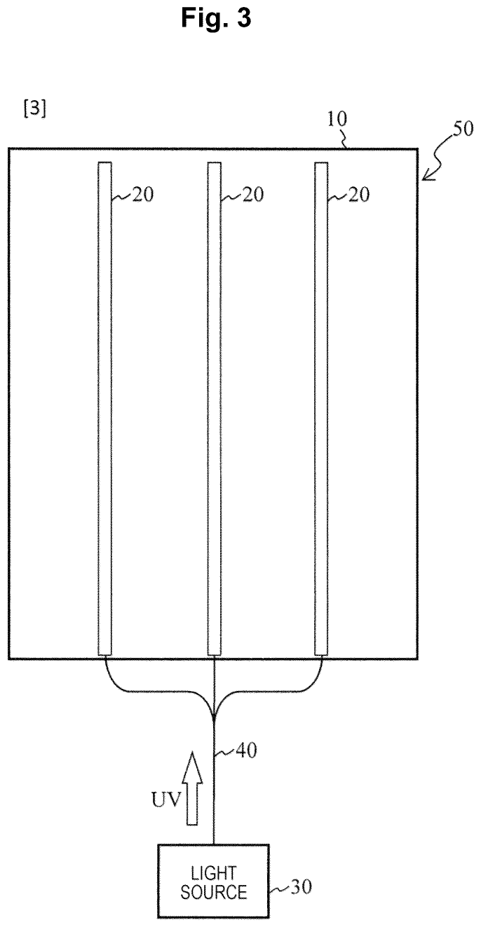

FIG. 3 is a diagram illustrating a light irradiation system including the photocatalytic sheet 50 according to the present embodiment. The light irradiation system includes a light source 30 that outputs ultraviolet light, the photocatalytic sheet 50, and an optical fiber 40 that transmits ultraviolet light from the light source 30 to the photocatalytic sheet 50.

When the optical fiber 20 has a structure of emitting ultraviolet light from the side surface as described with reference to FIG. 1, optical fibers 20 are disposed in parallel to the surface of the sheet 10 at regular intervals as illustrated in FIG. 3. The ultraviolet light output from the light source 30 is delivered to the optical fibers 20 through the optical fiber 40 and emitted to the photocatalytic layer 17. Disposing the light source 30 near the photocatalytic sheet 50 and supplying the ultraviolet light can enhance the reactions of the photocatalyst and lead to continuous reactions.

In FIG. 3, three optical fibers 20 are illustrated, but the number is not limited to three. The optical fibers 20 are disposed in parallel, but the fiber layout is not limited to a parallel layout. The system illustrated in FIG. 3 includes one light source 30 and the optical fiber 40 branches into the optical fibers 20 to deliver ultraviolet light, but the optical fibers 20 may be provided with respective light sources.

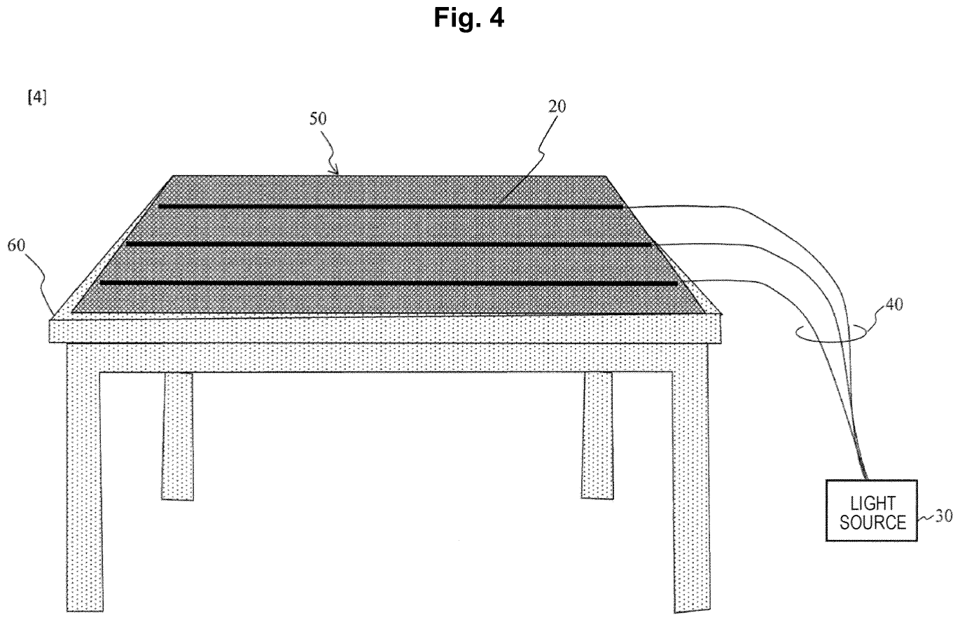

FIG. 4 is a diagram illustrating an example in which the light irradiation system described with reference to FIG. 3 is implemented by a table sheet. The table sheet (photocatalytic sheet 50) includes optical fibers and is coated with a photocatalyst, and is disposed on a top surface of a table 60. The table sheet is supplied with ultraviolet light from the light source 30, thereby enabling the photocatalytic reactions to occur without sunlight or illumination light.

### EXAMPLE 2

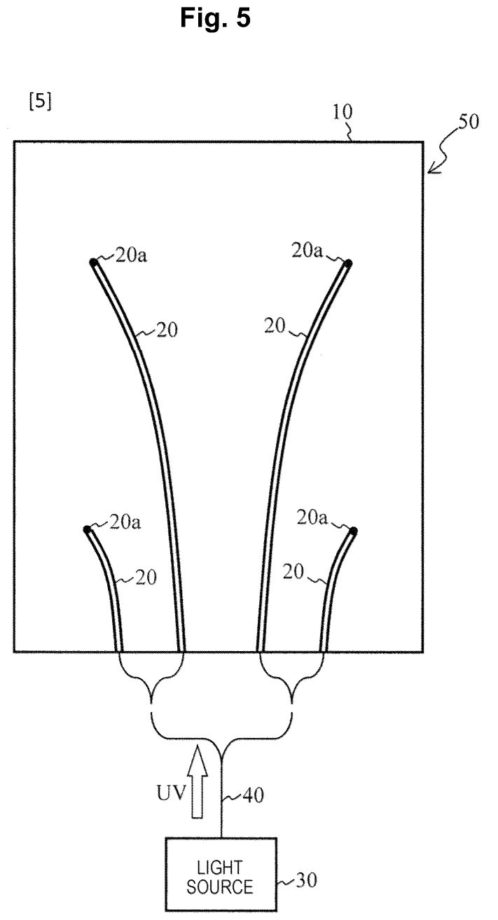

FIG. 5 is a diagram illustrating another light irradiation system including the photocatalytic sheet 50 according to the present embodiment. The light irradiation system illustrated in FIG. 5 differs from the light irradiation system illustrated in FIGS. 3 and 4 in that the system includes the photocatalytic sheet 50 illustrated in FIG. 2.

When optical fibers 20 are configured to emit ultraviolet light from the end 20a as in the structure described with reference to FIG. 2, the optical fibers 20 are disposed such that the ends 20a are located in separate positions from one another relative to the surface of the sheet 10 as illustrated in FIG. 5. The ultraviolet light output from the light source 30 is delivered to the optical fibers 20 through the optical fiber 40 and emitted to the photocatalytic layer 17.

In FIG. 5, four optical fibers 20 are illustrated, but the number is not limited to four. The ends 20a of the optical fibers 20 may be located at any positions. The system illustrated in FIG. 5 includes one light source 30 and the optical fiber 40 branches into the optical fibers 20 to deliver ultraviolet light, but the optical fibers 20 may be provided with respective light sources.

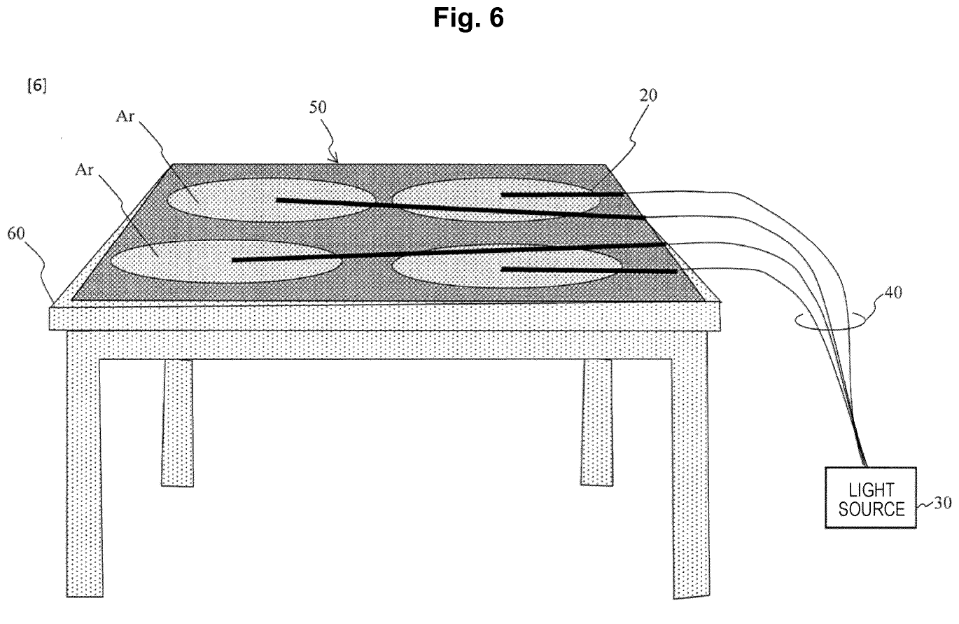

FIG. 6 is a diagram illustrating an example in which the light irradiation system described with reference to FIG. 4 is implemented by a table sheet. The table sheet (photocatalytic sheet 50) includes optical fibers and is coated with a photocatalyst, and is disposed on a top surface of a table 60. The table sheet is supplied with ultraviolet light from the light source 30, and the ultraviolet light is diffused radially from the end 20a of each optical fiber 20. Reference signs Ar in FIG. 6 denotes diffusion areas of ultraviolet light. The light irradiation system enables photocatalytic reactions to occur without sunlight or illumination light.

### (Effects)

Since the ultraviolet light dosage is inversely proportional to the square of the distance, irradiating the photocatalytic layer 17 with ultraviolet light at a close irradiation distance, which is achieved by the photocatalytic sheet 50, enables photocatalytic reactions to occur with a small output of ultraviolet light. Moreover, with the UV-A type ultraviolet light, which is harmless to the human body, serving as the ultraviolet light, constant irradiation is possible.

The light irradiation system including the photocatalytic sheet 50 can be freely designed in terms of the number, the intervals, and the like of the optical fibers 20 in accordance with the output of the light source 30, the irradiation capability of the optical fibers 20, the reactions and effects of the photocatalytic layer 17, and the like.

### REFERENCE SIGNS LIST

10 Sheet

17 Photocatalytic layer

20 Optical fiber

20a End

30 Light source

40 Optical fiber

50 Photocatalytic sheet

60 Table

1. A photocatalytic sheet comprising:
a sheet having a photocatalytic layer on one surface; and
an optical fiber embedded in the sheet and configured to supply ultraviolet light from inside of the sheet to the photocatalytic layer.

2. The photocatalytic sheet according to claim 1, wherein the optical fiber emits part of propagating ultraviolet light from a side surface.

3. The photocatalytic sheet according to claim 1, wherein
the optical fiber emits propagating ultraviolet light from an end,
the photocatalytic sheet further comprising a turning function embedded in the sheet and configured to direct the ultraviolet light emitted from the end of the optical fiber toward the photocatalytic layer.
